(19) United States
(12) Patent Application Publication
(10) Pub. No.: US 2014/0107487 A1
(43) Pub. Date: Apr. 17, 2014

(54) ULTRASOUND SYSTEM AND METHOD FOR AUTOMATICALLY ACTIVATING ULTRASOUND PROBE BASED ON MOTION OF THE ULTRASOUND PROBE

(71) Applicant: SAMSUNG MEDISON CO., LTD., Gangwon-do (KR)

(72) Inventors: Yong-soo KIM, Gangwon-do (KR); Jae-sung PARK, Gangwon-do (KR); Mi-ran SONG, Gangwon-do (KR); Seung-ju LEE, Gangwon-do (KR); Dong-gyu HYUN, Gangwon-do (KR)

(73) Assignee: SAMSUNG MEDISON CO., LTD., Gangwon-do (KR)

(21) Appl. No.: 14/052,587

(22) Filed: Oct. 11, 2013

(30) Foreign Application Priority Data
Oct. 11, 2012 (KR) 10-2012-0112799

(51) Int. Cl. A61B 8/00

(52) U.S. Cl. CPC A61B 8/4254; A61B 8/4444; A61B 8/4438; A61B 8/54; A61B 8/4477; A61B 8/461; A61B 8/56
USPC 600/459

(57) ABSTRACT

Provided are an ultrasound system and method for automatically activating an ultrasound probe based on motion of the ultrasound probe. The ultrasound system includes an ultrasound probe for transmitting an ultrasound signal to an object and receiving an ultrasound echo signal reflected from the object; a sensing unit for sensing the ultrasound probe and generating sensing information corresponding to motion of the ultrasound probe; and an automatic activation unit for activating the ultrasound probe based on the sensing information.

### RELATED APPLICATION

This application claims the benefit of Korean Patent Application No. 10-2012-112799, filed on Oct. 11, 2012, in the Korean Intellectual Property Office, the disclosure of which is incorporated herein in its entirety by reference.

### BACKGROUND

1. Field

One or more embodiments of the present invention relate to an ultrasound system, and more particularly, to an ultrasound system and method for activating an ultrasound probe based on motion of the ultrasound probe.

2. Description of the Related Art

Due to noninvasive and nondestructive characteristics, an ultrasound system is broadly used in the medical field to obtain information about inside an object. Also, the ultrasound system may provide a high-resolution image of tissues inside an object to a doctor without a surgical operation for directly making an incision in the object, and thus is often used in the medical field.

An ultrasound system includes at least one ultrasound probe for transmitting an ultrasound signal to an object and receiving an ultrasound signal reflected from the object (i.e., an ultrasound echo signal).

Conventionally, in order to activate a desired ultrasound probe, a user selects the ultrasound probe by using a user input unit such as a control panel. As such, whenever the desired ultrasound probe is changed, the ultrasound probe should be newly selected.

In order to solve the above problem, a pressure sensor is mounted on the ultrasound probe, a pressure applied to the ultrasound probe is sensed, and thus the ultrasound probe is activated. However, in order to activate the ultrasound probe, a pressure should be accurately applied to the pressure sensor mounted on the ultrasound probe.

### SUMMARY

One or more embodiments of the present invention include an ultrasound system and method for automatically activating an ultrasound probe based on motion of the ultrasound probe.

Additional aspects will be set forth in part in the description which follows and, in part, will be apparent from the description, or may be learned by practice of the presented embodiments.

According to one or more embodiments of the present invention, an ultrasound system includes an ultrasound probe for transmitting an ultrasound signal to an object and receiving an ultrasound echo signal reflected from the object; a sensing unit for sensing the ultrasound probe and generating sensing information corresponding to motion of the ultrasound probe; and an automatic activation unit for activating the ultrasound probe based on the sensing information.

The ultrasound probe may include an identification information unit for storing identification information.

The identification information unit may include a radio frequency identification (RFID) tag.

The identification information unit may be mounted on an inner or outer side of the ultrasound probe in a form of a sticker.

The identification information unit may be mounted on an outer side of the ultrasound probe in a detachable form.

The sensing unit may determine whether the identification information is readable, by sensing the identification information unit; may read the identification information from the identification information unit when it is determined that the identification information is readable; may generate first sensing information including the read identification information; and may generate second sensing information corresponding to the motion of the ultrasound probe when it is determined that the identification information is not readable, and the second sensing information may not include the identification information.

The sensing unit may include an RFID reader.

The automatic activation unit may include a storage unit for storing a mapping table for providing the identification information corresponding to the ultrasound probe; a relay unit for activating the ultrasound probe by connecting the ultrasound probe; and a control unit for generating a control signal for activating the ultrasound probe by driving the relay unit based on the sensing information.

The control unit may determine whether the second sensing information exists, by analyzing the sensing information provided from the sensing unit; may detect the ultrasound probe corresponding to the second sensing information by searching the storage unit, when it is determined that the second sensing information exists; and may generate the control signal for activating the detected ultrasound probe by driving the relay unit.

The control unit may detect the ultrasound probe corresponding to the second sensing information by comparing the identification information included in the first sensing information to the identification information of the mapping table.

The sensing unit may determine whether the identification information is readable, by sensing the identification information unit; may read the identification information from the identification information unit when it is determined that the identification information is readable; may generate first sensing information including the read identification information; and may not generate the sensing information when it is determined that the identification information is not readable.

The automatic activation unit may include a storage unit for storing a mapping table for providing the identification information corresponding to the ultrasound probe; a relay unit for activating the ultrasound probe by connecting the ultrasound probe; and a control unit for generating a control signal for activating the ultrasound probe by driving the relay unit based on the sensing information.

The control unit may determine whether the first sensing information is provided from the sensing unit; may detect the ultrasound probe that does not provide the first sensing information, by searching the storage unit, when it is determined that the first sensing information is not provided; and may generate the control signal for activating the detected ultrasound probe by driving the relay unit.

The sensing unit may include a sensor for sensing the identification information unit; and an antenna for reading and transmitting the identification information stored in the identification information unit.

The antenna may be disposed at a side of a holder for holding the ultrasound probe.

The antenna may be disposed in such a way that sensing regions overlap each other.

The ultrasound probe may include a magnetic field generator.

The sensing unit may include an antenna including a coil for generating an induced current by sensing the magnetic field generator.

The antenna may be disposed at a side of a holder for holding the ultrasound probe.

The antenna may be disposed in such a way that sensing regions overlap each other.

The automatic activation may include a storage unit for storing a mapping table for providing information on the sensing unit corresponding to the ultrasound probe; a relay unit for activating the ultrasound probe by connecting the ultrasound probe; and a control unit for generating a control signal for activating the ultrasound probe by driving the relay unit based on the sensing information.

The control unit may detect the ultrasound probe corresponding to the sensing unit by searching the storage unit, when the sensing information is provided from the sensing unit; and may connect the detected ultrasound probe and generates the control signal for activating the detected ultrasound probe.

According to one or more embodiments of the present invention, a method of activating an ultrasound probe includes a) sensing the ultrasound probe and generating sensing information corresponding to motion of the ultrasound probe; and b) activating the ultrasound probe based on the sensing information.

The step a) may include determining whether an identification information is readable, by sensing the ultrasound probe including an identification information unit for storing the identification information; reading the identification information from the identification information unit when it is determined that the identification information is readable; generating first sensing information including the read identification information; and generating second sensing information corresponding to the motion of the ultrasound probe when it is determined that the identification information is not readable, and the second sensing information may not include the identification information.

The step b) may include b1) determining whether the second sensing information exists, by analyzing the sensing information; b2) detecting the ultrasound probe corresponding to the second sensing information by searching a storage unit for storing a mapping table for providing the identification information corresponding to the ultrasound probe, when it is determined that the second sensing information exists; and b3) generating a control signal for activating the detected ultrasound probe.

The step b2) may include detecting the ultrasound probe corresponding to the second sensing information by comparing the identification information included in the first sensing information to the identification information of the mapping table.

The step a) may include determining whether the identification information is readable, by sensing the ultrasound probe including an identification information unit for storing the identification information; reading the identification information from the identification information unit when it is determined that the identification information is readable; generating first sensing information including the read identification information; and not generating the sensing information when it is determined that the identification information is not readable.

The step b) may include determining whether the first sensing information is provided; detecting the ultrasound probe that does not provide the first sensing information, by searching a storage unit for storing a mapping table for providing the identification information corresponding to the ultrasound probe, when it is determined that the first sensing information is not provided; and generating a control signal for activating the detected ultrasound probe.

The step a) may include generating an induced current based on a magnetic field generated by the ultrasound probe including a magnetic field generator; and generating the sensing information based on the induced current.

### BRIEF DESCRIPTION OF THE DRAWINGS

These and/or other aspects will become apparent and more readily appreciated from the following description of the embodiments, taken in conjunction with the accompanying drawings of which:

FIG. 1 is a block diagram of an ultrasound system according to an embodiment of the present invention;

FIG. 2 is a schematic diagram of a control panel, an ultrasound probe, an ultrasound probe holder, and a sensor, according to an embodiment of the present invention; and

FIG. 3 is a block diagram of an automatic activation unit according to an embodiment of the present invention.

### DETAILED DESCRIPTION

All terms including descriptive or technical terms which are used herein should be construed as having meanings that are obvious to one of ordinary skill in the art. However, the terms may have different meanings according to an intention of one of ordinary skill in the art, precedent cases, or the appearance of new technologies. In addition, some terms may be arbitrarily selected by the applicant, and in this case, the meaning of the selected terms will be described in detail in the detailed description. Thus, the terms used herein should be defined based on the meaning of the terms together with the description throughout the specification.

Further, when a part “includes” or “comprises” an element, unless there is a particular description contrary thereto, the part can further include other elements, not excluding the other elements. In the following description, terms such as “unit” and “module” indicate a unit for processing at least one function or operation, wherein the unit and the block may be embodied as hardware or software or embodied by combining hardware and software.

Throughout the specification, “an ultrasound image” refers to an image which is obtained from an object by using ultrasonic waves. The object may refer to a part of the body. For example, the object may include an organ such as any one or more of a liver, a heart, a uterus, a brain, a breast, or an abdomen, or a fetus.

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

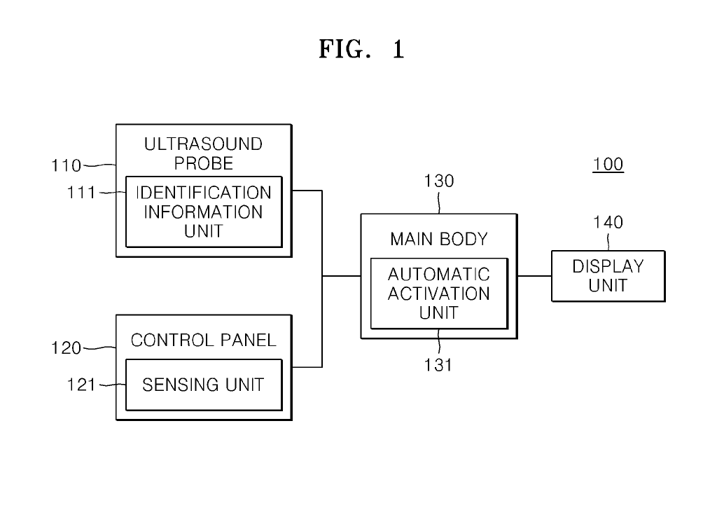

FIG. 1 is a block diagram of an ultrasound system 100 according to an embodiment of the present invention. Referring to FIG. 1, the ultrasound system 100 includes an ultrasound probe 110.

The ultrasound probe 110 transmits an ultrasound signal to an object, receives an ultrasound signal reflected from the object (i.e., an ultrasound echo signal), and generates an electrical signal (a reception signal). The ultrasound probe 110 is connected to a main body 130 of the ultrasound system 100 by wire or wirelessly. The ultrasound probe 110 includes a convex probe, a linear probe, a three-dimensional probe, a phased array probe, etc.

According to an embodiment, the ultrasound probe 110 includes an identification information unit 111. The identification information unit 111 stores identification information for identifying the ultrasound probe 110. The identification information unit 111 includes a radio frequency identification (RFID) tag. However, the identification information unit 111 is not limited thereto. The identification information unit 111 is mounted on an inner or outer side of the ultrasound probe 110 in various forms. For example, the identification information unit 111 may be mounted on an inner or outer side of the ultrasound probe 110 in the form of an attachable sticker. As another example, the identification information unit 111 may be mounted on an outer side of the ultrasound probe 110 in a detachable form (for example, in the form of a detachable accessory such as a ring).

According to another embodiment, the ultrasound probe 110 includes a magnetic field generator (not shown). Like the identification information unit 111, the magnetic field generator may be mounted on an inner or outer side of the ultrasound probe 110 in various forms.

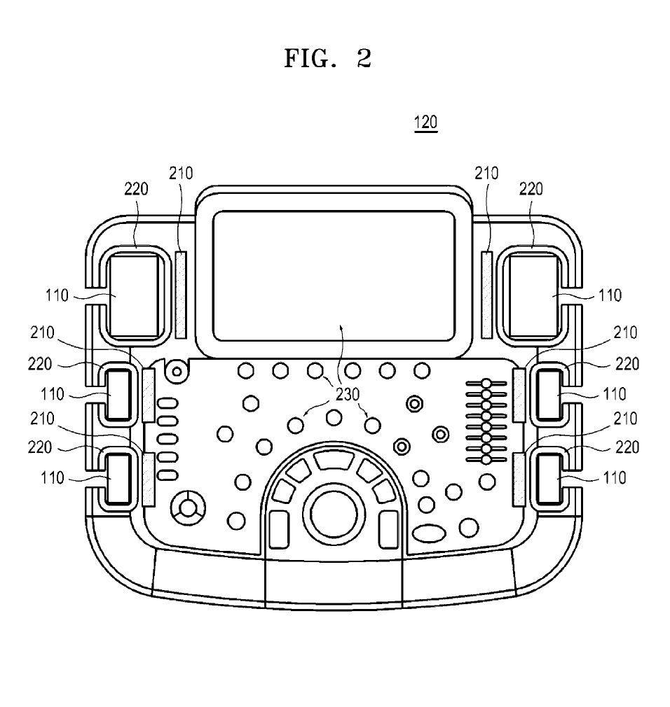

Referring back to FIG. 1, the ultrasound system 100 further includes a control panel 120. The control panel 120 is connected to the main body 130 of the ultrasound system 100 by wire or wirelessly. The control panel 120 includes a sensing unit 121. Also, as illustrated in FIG. 2, the control panel 120 further includes an ultrasound probe holder 220 for holding the ultrasound probe 110, and a user input unit 230 for receiving input information of a user. The user input unit 230 includes a plurality of buttons, a plurality of slices, a keyboard, a trackball, etc.

The sensing unit 121 senses motion of the ultrasound probe 110 and generates sensing information corresponding to the motion of the ultrasound probe 110.

According to an embodiment, the sensing unit 121 may include a sensor (not shown) for sensing the identification information unit 111 of the ultrasound probe 110 in a preset time cycle, a first antenna 210 (see FIG. 2) for reading and transmitting the identification information stored in the identification information unit 111 of the ultrasound probe 110, and a first sensing information generator (not shown) for generating the sensing information corresponding to the motion of the ultrasound probe 110 based on whether the identification information is readable. The sensing unit 121 includes an RFID reader. However, the sensing unit 121 is not limited thereto. Also, the first antenna 210 is mounted at a position for sensing the identification information unit 111 of the ultrasound probe 110 held by the ultrasound probe holder 220. For example, as illustrated in FIG. 2, the first antenna 210 is mounted at a position spaced apart from the ultrasound probe holder 220 by a preset distance. Also, the first antenna 210 may be mounted in such a way that regions for sensing a plurality of ultrasound probes 110 (i.e., the identification information units 111 of the ultrasound probes 110) overlap each other and thus the ultrasound probes 110 are not simultaneously activated. The first antenna 210 may be mounted in such a way that the regions for sensing the ultrasound probes 110 (i.e., the identification information units 111 of the ultrasound probes 110) do not overlap each other.

For example, the sensing unit 121 senses the identification information unit 111 of the ultrasound probe 110 in a preset time cycle and determines whether the identification information stored in the identification information unit 111 of the ultrasound probe 110 is readable. When it is determined that the identification information stored in the identification information unit 111 of the ultrasound probe 110 is readable, that is, if the ultrasound probe 110 is held by the ultrasound probe holder 220 and thus it is determined that the identification information unit 111 of the ultrasound probe 110 is at a position sensible by the sensing unit 121, the sensing unit 121 reads the identification information from the identification information unit 111, and generates the sensing information including the read identification information (hereinafter referred to as first sensing information). Otherwise, when it is determined that the identification information stored in the identification information unit 111 of the ultrasound probe 110 is not readable, that is, if the ultrasound probe 110 is separated from the ultrasound probe holder 220 and thus it is determined that the ultrasound probe 110 is out of the position sensible by the sensing unit 121, the sensing unit 121 generates the sensing information indicating that the ultrasound probe 110 is separated from the ultrasound probe holder 220 (hereinafter referred to as second sensing information). In other words, the second sensing information is the sensing information corresponding to the motion of the ultrasound probe 110.

As another example, the sensing unit 121 senses the identification information unit 111 of the ultrasound probe 110 in a preset time cycle and determines whether the identification information stored in the identification information unit 111 of the ultrasound probe 110 is readable. When it is determined that the identification information stored in the identification information unit 111 of the ultrasound probe 110 is readable, that is, if the ultrasound probe 110 is held by the ultrasound probe holder 220 and thus it is determined that the identification information unit 111 of the ultrasound probe 110 is at a position sensible by the sensing unit 121, the sensing unit 121 reads the identification information from the identification information unit 111 of the ultrasound probe 110, and generates the first sensing information including the read identification information. Otherwise, when it is determined that the identification information stored in the identification information unit 111 of the ultrasound probe 110 is not readable, that is, if the ultrasound probe 110 is separated from the ultrasound probe holder 220 and thus it is determined that the ultrasound probe 110 is out of the position sensible by the sensing unit 121, the sensing unit 121 does not generate the sensing information. In other words, the sensing unit 121 generates the sensing information only when the identification information of the identification information unit 111 is readable.

According to another embodiment, the sensing unit 121 includes a second antenna (not shown) including a coil (not shown) for sensing the ultrasound probe 110 (i.e., the magnetic field generator of the ultrasound probe 110) and generating an induced current, and a second sensing information generator (not shown) for generating the sensing information corresponding to the motion of the ultrasound probe 110 based on the induced current. The second antenna is mounted on a side of the control panel 120. In more detail, the second antenna may be mounted at a position for allowing the coil to generate the induced current whenever the coil is included in a magnetic field generated by the magnetic field generator of the ultrasound probe 110. The second antenna may be mounted in such a way that regions for sensing a plurality of ultrasound probes 110 (i.e., the identification information units 111 of the ultrasound probes 110) overlap each other and thus the ultrasound probes 110 are not simultaneously activated. The second antenna may be mounted in such a way that the regions for sensing the ultrasound probes 110 (i.e., the identification information units 111 of the ultrasound probes 110) do not overlap each other.

For example, if the ultrasound probe 110 is included in a sensing region of the second antenna, that is, if a coil of the sensing unit 121 is included in the magnetic field generated by the magnetic field generator of the ultrasound probe 110, the sensing unit 121 (i.e., the coil of the sensing unit 121) generates an induced current, and generates the sensing information corresponding to the motion of the ultrasound probe 110 based on the generated induced current. Otherwise, if the ultrasound probe 110 is not included in the sensing region of the second antenna, that is, if the coil of the sensing unit 121 is not included in the magnetic field generated by the magnetic field generator of the ultrasound probe 110, the sensing unit 121 does not generate the sensing information. In other words, the sensing unit 121 generates the sensing information only when the motion of the ultrasound probe 110 occurs (that is, only when the ultrasound probe 110 is separated from the ultrasound probe holder 220).

Referring back to FIG. 1, the ultrasound system 100 further includes the main body 130. The main body 130 includes an automatic activation unit 131. Also, the main body 130 further includes a transmission unit (not shown) for generating a transmission signal for obtaining an ultrasound image, a reception unit (not shown) for generating a digital signal by performing analog-digital conversion on the reception signal provided from the ultrasound probe 110, and generating a reception focusing signal by performing beam forming on the digital signal, an ultrasound data generator (not shown) for generating ultrasound data corresponding to the ultrasound image by using the reception focusing signal, an image generator (not shown) for generating the ultrasound image corresponding to the ultrasound data, etc.

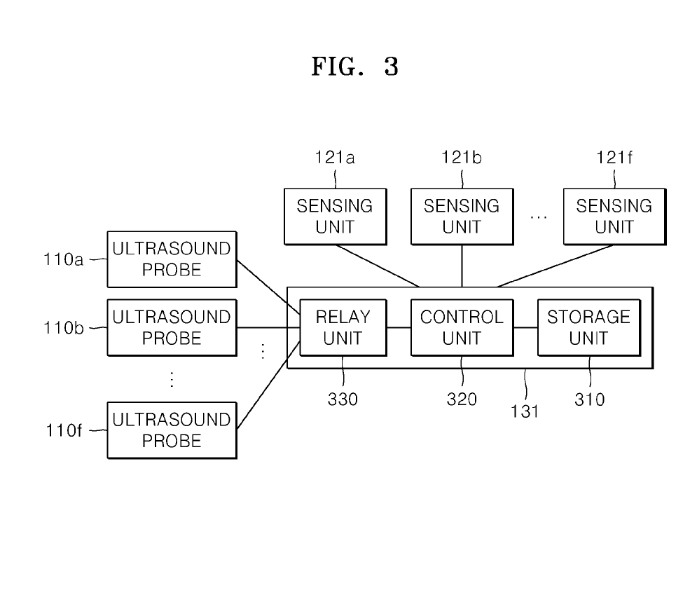

FIG. 3 is a block diagram of the automatic activation unit 131 according to an embodiment of the present invention. In FIG. 3, for convenience of explanation, it is assumed that first through sixth ultrasound probes 110a through 110f are connected to the main body 130 as the ultrasound probe 110, and that first through sixth sensing units 121a through 121f corresponding one-to-one to the first through sixth ultrasound probes 110a through 110f are mounted on the control panel 120 as the sensing unit 121. Referring to FIG. 3, the automatic activation unit 131 includes a storage unit 310, a control unit 320, and a relay unit 330.

The storage unit 310 stores information for automatically activating the ultrasound probe 110 (hereinafter referred to as automatic activation information). For example, the storage unit 310 stores a mapping table for providing identification information corresponding to each of the first through sixth ultrasound probes 110a through 110f, as shown in Table 1.

TABLE 1

| Ultrasound Probe | Identification Information |
|---|---|
| first ultrasound probe 110a | first identification information |
| second ultrasound probe 110b | second identification information |
| third ultrasound probe 110c | third identification information |
| fourth ultrasound probe 110d | fourth identification information |
| fifth ultrasound probe 110e | fifth identification information |
| sixth ultrasound probe 110f | sixth identification information |

As another example, the storage unit 310 stores a mapping table for providing information on the first through sixth sensing units 121a through 121f corresponding one-to-one to the first through sixth ultrasound probes 110a through 110f, as shown in Table 2.

TABLE 2

| Ultrasound Probe | Sensing Unit |
|---|---|
| first ultrasound probe 110a | first sensing unit 121a |
| second ultrasound probe 110b | second sensing unit 121b |
| third ultrasound probe 110c | third sensing unit 121c |
| fourth ultrasound probe 110d | fourth sensing unit 121d |
| fifth ultrasound probe 110e | fifth sensing unit 121e |
| sixth ultrasound probe 110f | sixth sensing unit 121f |

The control unit 320 generates a control signal for activating the ultrasound probe 110 based on the sensing information received from the first through sixth sensing units 121a through 121f and the mapping table stored in the storage unit 310.

According to an embodiment, the control unit 320 receives the sensing information provided from each of the first through sixth sensing units 121a through 121f in a preset time cycle. The control unit 320 generates the control signal for activating the ultrasound probe 110 based on the received sensing information and the mapping table stored in the storage unit 310.

For example, the control unit 320 analyzes the sensing information provided from the first through sixth sensing units 121a through 121f, and determines whether the second sensing information not including the identification information exists. When it is determined that the second sensing information exists, the control unit 320 detects an ultrasound probe corresponding to the second sensing information by searching the storage unit 310. In other words, the control unit 320 detects the ultrasound probe corresponding to the second sensing information by comparing the identification information included in the received sensing information to the identification information of the mapping table stored in the storage unit 310. The control unit 320 drives the relay unit 330 to connect the detected ultrasound probe to the main body 130, and generates the control signal for activating the detected ultrasound probe. Otherwise, when it is determined that the second sensing information does not exist, the control unit 320 does not generate the control signal.

For example, if the second sensing information is provided from the first sensing unit 121a, and the first sensing information is provided from the second through sixth sensing units 121b through 121f, the control unit 320 detects the first ultrasound probe 110a as the ultrasound probe corresponding to the second sensing information, by comparing the identification information of the provided first sensing information to the identification information of the mapping table stored in the storage unit 310. The control unit 320 drives the relay unit 330 to connect the first ultrasound probe 110a to the main body 130, and generates the control signal for activating the first ultrasound probe 110a.

As another example, the control unit 320 determined whether the first sensing information is provided from the first through sixth sensing units 121a through 121f, in a preset time cycle. When it is determined that the first sensing information is not provided from any one of the first through sixth sensing units 121a through 121f, the control unit 320 determines an ultrasound probe not corresponding to the first sensing information by comparing the identification information included in the provided first sensing information to the identification information of the mapping table stored in the storage unit 310. In other words, the control unit 320 detects an ultrasound probe corresponding to a sensing unit that does not provide the first sensing information. The control unit 320 drives the relay unit 330 to connect the detected ultrasound probe to the main body 130, and generates the control signal for activating the detected ultrasound probe. Otherwise, when it is determined that the first sensing information is provided from all of the first through sixth sensing units 121a through 121f, the control unit 320 does not generate the control signal.

For example, if the first sensing information is not provided from the first sensing unit 121a, and the first sensing information is provided from the second through sixth sensing units 121a through 121f, the control unit 320 detects the first ultrasound probe 110a not corresponding to the first sensing information, by comparing the identification information of the provided first sensing information to the identification information of the mapping table stored in the storage unit 310. In other words, the control unit 320 detects the first ultrasound probe 110a corresponding to the first sensing unit 121a that does not provide the first sensing information. The control unit 320 drives the relay unit 330 to connect the first ultrasound probe 110a to the main body 130, and generates the control signal for activating the first ultrasound probe 110a.

According to another embodiment, the control unit 320 receives the sensing information provided from the first through sixth sensing units 121a through 121f. The control unit 320 generates the control signal for activating the ultrasound probe 110 based on the received sensing information and the mapping table stored in the storage unit 310.

For example, if the sensing information is provided from the first sensing unit 121a, and the sensing information is not provided from the second through sixth sensing units 121b through 121f, the control unit 320 compares the provided sensing information to the mapping table stored in the storage unit 310 (i.e., information on sensing units included in the mapping table), and detects the first ultrasound probe 110a corresponding to the first sensing unit 121a that provides the sensing information, as an ultrasound probe to be activated. The control unit 320 drives the relay unit 330 to connect the first ultrasound probe 110a to the main body 130, and generates the control signal for activating the first ultrasound probe 110a.

Referring back to FIG. 3, the relay unit 330 connects the ultrasound probe 110 to the main body 130 based on the control signal provided from the control unit 320. The relay unit 330 may be any device capable of connecting the ultrasound probe 110 to the main body 130 based on the control signal of the control unit 320.

Referring back to FIG. 1, the ultrasound system 100 further includes a display unit 140. The display unit 140 displays the ultrasound image generated by the main body 130. The display unit 140 includes a liquid crystal display (LCD), a light emitting diode (LED), etc.

As described above, according to one or more of the above embodiments of the present invention, a desired ultrasound probe may be automatically activated by sensing motion of the ultrasound probe, and thus convenience may be provided to a user.

Also, the ultrasound probe may be automatically activated by sensing the motion of the ultrasound probe without accurately sensing a contact of a user.

Furthermore, an existing ultrasound probe may be used by mounting an identification information unit including identification information on the ultrasound probe without additionally mounting a sensor.

While one or more embodiments of the present invention have been described with reference to the figures, it will be understood by those of ordinary skill in the art that various changes in form and details may be made therein without departing from the spirit and scope of the present invention as defined by the following claims.

For example, although the first antenna 210 and the ultrasound probe holder 220 are mounted on the control panel 120 in the above description, the first antenna 210 and the ultrasound probe holder 220 are not limited thereto and may be mounted on the main body 130 or the display unit 140.

What is claimed is:

1. An ultrasound system comprising:
an ultrasound probe for transmitting an ultrasound signal to an object and receiving an ultrasound echo signal reflected from the object;
a sensing unit for sensing the ultrasound probe and generating sensing information corresponding to motion of the ultrasound probe; and
an automatic activation unit for activating the ultrasound probe based on the sensing information.

2. The ultrasound system of claim 1, wherein the ultrasound probe comprises an identification information unit for storing identification information.

3. The ultrasound system of claim 2, wherein the identification information unit comprises a radio frequency identification (RFID) tag.

4. The ultrasound system of claim 2, wherein the identification information unit is mounted on an inner or outer side of the ultrasound probe in a form of a sticker.

5. The ultrasound system of claim 2, wherein the identification information unit is mounted on an outer side of the ultrasound probe in a detachable form.

6. The ultrasound system of claim 2, wherein the sensing unit:
determines whether the identification information is readable, by sensing the identification information unit;
reads the identification information from the identification information unit when it is determined that the identification information is readable;
generates first sensing information including the read identification information; and
generates second sensing information corresponding to the motion of the ultrasound probe when it is determined that the identification information is not readable, and
wherein the second sensing information does not include the identification information.

7. The ultrasound system of claim 6, wherein the sensing unit comprises an RFID reader.

8. The ultrasound system of claim 6, wherein the automatic activation unit comprises:
a storage unit for storing a mapping table for providing the identification information corresponding to the ultrasound probe;
a relay unit for activating the ultrasound probe by connecting the ultrasound probe; and
a control unit for generating a control signal for activating the ultrasound probe by driving the relay unit based on the sensing information.

9. The ultrasound system of claim 8, wherein the control unit:
determines whether the second sensing information exists, by analyzing the sensing information provided from the sensing unit;
detects the ultrasound probe corresponding to the second sensing information by searching the storage unit, when it is determined that the second sensing information exists; and
generates the control signal for activating the detected ultrasound probe by driving the relay unit.

10. The ultrasound system of claim 9, wherein the control unit detects the ultrasound probe corresponding to the second sensing information by comparing the identification information included in the first sensing information to the identification information of the mapping table.

11. The ultrasound system of claim 2, wherein the sensing unit:
determines whether the identification information is readable, by sensing the identification information unit;
reads the identification information from the identification information unit when it is determined that the identification information is readable;
generates first sensing information including the read identification information; and
does not generate the sensing information when it is determined that the identification information is not readable.

12. The ultrasound system of claim 11, wherein the automatic activation unit comprises:
a storage unit for storing a mapping table for providing the identification information corresponding to the ultrasound probe;
a relay unit for activating the ultrasound probe by connecting the ultrasound probe; and
a control unit for generating a control signal for activating the ultrasound probe by driving the relay unit based on the sensing information.

13. The ultrasound system of claim 12, wherein the control unit:
determines whether the first sensing information is provided from the sensing unit;
detects the ultrasound probe that does not provide the first sensing information, by searching the storage unit, when it is determined that the first sensing information is not provided; and
generates the control signal for activating the detected ultrasound probe by driving the relay unit.

14. The ultrasound system of claim 2, wherein the sensing unit comprises:
a sensor for sensing the identification information unit; and
an antenna for reading and transmitting the identification information stored in the identification information unit.

15. The ultrasound system of claim 14, wherein the antenna is disposed at a side of a holder for holding the ultrasound probe.

16. The ultrasound system of claim 14, wherein the antenna is disposed in such a way that sensing regions overlap each other.

17. The ultrasound system of claim 2, wherein the ultrasound probe comprises a magnetic field generator.

18. The ultrasound system of claim 17, wherein the sensing unit comprises an antenna including a coil for generating an induced current by sensing the magnetic field generator.

19. The ultrasound system of claim 18, wherein the antenna is disposed at a side of a holder for holding the ultrasound probe.

20. The ultrasound system of claim 18, wherein the antenna is disposed in such a way that sensing regions overlap each other.

21. The ultrasound system of claim 18, wherein the automatic activation comprises:
a storage unit for storing a mapping table for providing information on the sensing unit corresponding to the ultrasound probe;
a relay unit for activating the ultrasound probe by connecting the ultrasound probe; and
a control unit for generating a control signal for activating the ultrasound probe by driving the relay unit based on the sensing information.

22. The ultrasound system of claim 21, wherein the control unit:
detects the ultrasound probe corresponding to the sensing unit by searching the storage unit, when the sensing information is provided from the sensing unit; and
connects the detected ultrasound probe and generates the control signal for activating the detected ultrasound probe.

23. A method of activating an ultrasound probe, the method comprising:
a) sensing the ultrasound probe and generating sensing information corresponding to motion of the ultrasound probe; and
b) activating the ultrasound probe based on the sensing information.

24. The method of claim 23, wherein the step a) comprises:
determining whether an identification information is readable, by sensing the ultrasound probe comprising an identification information unit for storing the identification information;
reading the identification information from the identification information unit when it is determined that the identification information is readable;
generating first sensing information including the read identification information; and
generating second sensing information corresponding to the motion of the ultrasound probe when it is determined that the identification information is not readable, and
wherein the second sensing information does not include the identification information.

25. The method of claim 24, wherein the step b) comprises:
b1) determining whether the second sensing information exists, by analyzing the sensing information;
b2) detecting the ultrasound probe corresponding to the second sensing information by searching a storage unit for storing a mapping table for providing the identification information corresponding to the ultrasound probe, when it is determined that the second sensing information exists; and
b3) generating a control signal for activating the detected ultrasound probe.

26. The method of claim 25, wherein the step b2) comprises detecting the ultrasound probe corresponding to the second sensing information by comparing the identification information included in the first sensing information to the identification information of the mapping table.

27. The method of claim 23, wherein the step a) comprises:
determining whether the identification information is readable, by sensing the ultrasound probe comprising an identification information unit for storing the identification information;
reading the identification information from the identification information unit when it is determined that the identification information is readable;
generating first sensing information including the read identification information; and
not generating the sensing information when it is determined that the identification information is not readable.

28. The method of claim 27, wherein the step b) comprises:
determining whether the first sensing information is provided;
detecting the ultrasound probe that does not provide the first sensing information, by searching a storage unit for storing a mapping table for providing the identification information corresponding to the ultrasound probe, when it is determined that the first sensing information is not provided; and
generating a control signal for activating the detected ultrasound probe.

29. The method of claim 23, wherein the step a) comprises:
generating an induced current based on a magnetic field generated by the ultrasound probe comprising a magnetic field generator; and
generating the sensing information based on the induced current.
